(19) United States
(12) Patent Application Publication
(10) Pub. No.: US 2007/0162064 A1
(43) Pub. Date: Jul. 12, 2007

(54) Lancet casing

(76) Inventor: Charles D. Starnes, Coral Springs, FL (US)

Correspondence Address: ROGERS TOWERS, P.A., 1301 RIVERPLACE BOULEVARD, SUITE 1500, JACKSONVILLE, FL, 32207, US

(21) Appl. No.: 11/717,346

(22) Filed: Mar. 13, 2007

Related U.S. Application Data
(62) Division of application No. 10/364,782, filed on Feb. 11, 2003.
(60) Provisional application No. 60/364,478, filed on Mar. 15, 2002.

(51) Int. Cl. A61B 17/32

(52) U.S. Cl. 606/181

(57) ABSTRACT

An improved protective lancet, lancet casing and method for producing the same. The lancet casing includes a lancet body that is integrally molded about an elongated shaft portion of a lancet needle. The lancet body is characterized in having an upper bearing surface from which a pointed end of the lancet needle extends. The lancet casing further includes a cap integrally molded about the pointed end of the lancet needle and joined to the lancet body by a narrow frangible junction on the upper bearing surface of the lancet body. In accordance with the present invention, one or more tab members are affixed between the cap and the body. Each of the one or more tab members includes a persistently affixed upper end integrally molded to the outer contour of the cap and a detachably affixed lower end integrally molded to or near a peripheral edge of the upper bearing surface of the lancet body to provide a readily perceptible user indication of the status of whether or not the cap member has been re-attached or tampered with.

### CROSS REFERENCE TO RELATED APPLICATIONS

This application is a divisional of U.S. patent application Ser. No. 10/364,782, filed on Feb. 11, 2003, which claims the benefit of and priority from U.S. provisional patent application Ser. No. 60/364,478 filed on Mar. 15, 2002 and U.S. design patent application Ser. No. 29/171,683 filed on Nov. 26, 2002 and the contents of each is incorporated herein by this reference.

### BACKGROUND OF THE INVENTION

1. Technical Field

The present invention relates in general to the field of medical lancet devices that utilize protective caps for user safety and security. More particularly, the invention relates to such devices capable of covering the lancet needle to protect from unintended puncturing during storage and handling and to maintain sterility prior to use. Additionally, the invention relates to providing a method of assuring that lancets are limited to one-time use only. Specifically, the invention provides a method for indicating whether the lancet's protective cap has been removed and re-attached to prevent re-use and to detect tampering.

2. Description of the Related Art

Lancets and their uses are well known in the art as a convenient and safe means for piercing or pricking a person's skin to draw small amounts blood that can then be subjected to a variety of medical tests such as for blood sugar content. A typical integrated lancet design is as described in U.S. Pat. No. 3,358,689, with a lancet needle encased in an elongated plastic body. The lancet needle tip is embedded in a protective cap that is integrally molded with a plastic body to form a single integral unit prior to use.

Lancets are designed to draw a relatively minute quantity of blood from capillaries immediately below the surface of the skin and are commonly used by the patients themselves at home as well as by medical professionals in medical offices and hospitals. Lancets are used to prick the patient's skin either by manual penetration or in conjunction with a dynamic (usually spring-driven) mechanical device into which the lancet unit is installed and then removed after use. In the home setting, most lancets are used by the patient with the mechanical device, which is designed to accept lancets of various sizes. In the medical office and hospital setting, lancets are often used manually in order avoid the necessity of having to re-sterilize the mechanical device after each use.

Lancets are specifically designed to safely accommodate their purpose as “pricking” devices that are common utilized by non-medically trained individuals including children. Specifically, in order to effectuate a safe and controlled puncturing of a patient's skin, the exposed tip of the needle extends only a very short distance (typically a few millimeters) from the flat upper surface of the lancet body, which acts as a bearing to limit the puncture depth. The needle may extend from the upper surface using any well known lancet configurations, such as by having the needle protrude outward from the lancet body or by otherwise affixing the needle to the upper surface. Together, these signature structural and dimensional characteristics of lancet design give rise to another nearly universal lancet design feature, namely, a single unitary molded plastic casing that includes a plastic cap that covers the lancet tip. The cap is integrally molded with the lancet “body” which encases most of the needle shaft and provides the bearing surface as explained above. The cap is moldedly bonded to the top surface of the lancet body at a relatively narrow neck or seal boundary, such that a user can remove the cap with relative ease using a twisting motion just prior to use. In addition to its compatibility with the short needle/bearing structural design of lancets, the unitary plastic encasement promotes additional safety by providing reliable sterilization from the heat of injection molding and subsequent airtight encasement. These basic tenets of lancet design have consistently provided a safe and effective blood sampling means that has remained fundamentally unchanged for many years.

A problem arises, however, concerning safety after a lancet has been used to draw blood. After the lancet unit has been used to prick the patient's skin, either manually or by means of a mechanical lancing device, the sharp lancet tip is contaminated with the patient's blood. As with any instrument or device that has been exposed to blood, a used lancet unit constitutes a significant biological hazard that must be handled with care. Therefore, after use and prior to ultimate disposal of the lancet unit, the cover cap may be re-attached to prevent accidental contact by circumstantially vulnerable persons such as sanitation workers or janitors with the extremely sharp tip of the lancet.

However, the solid casing structure in which the cap and body form a single continuous outer casing presents difficulties relating to a user's ability to identify a re-attached lancet cap and therefore to distinguish a used from an unused lancet unit. A re-attached lancet cap is not easily recognizable because the cap is replaced in flush surface-to-surface abutment against the lancet body at a very small frangible junction necessitated by the need for relative ease of the original detachment. Consequently the re-attached cap/body appear as a continuous unit tending to obscure visual evidence of any prior disengagement and closely resembling the original unbreached unit. The foregoing general design characteristics of lancets also hinder a user's ability to identify a used lancet unit by “feel.” A re-attached cap is either snugly reinserted into the original molded cavity from which the needle was originally withdrawn, or, due to the difficultly encountered in attempting to insert the needle into the cap's miniscule extant cavity, the cap is forcibly impaled on the needle at a different surface location. The tight fit of the impaled cap combined with the friction offered by the semi-adhered plastic-on-plastic surface boundary between the cap and the lancet body result in subsequent cap “detachments” requiring a significant amount of force which may easily be mistaken as that required for the initial cap detachment. Thus, there is not always a materially noticeable difference between the look or feel of removing a lancet cap for the first time and the look or feel of subsequent detachments.

Since lancets are commonly stored in bags or boxes of more than fifty, the inability to readily and reliably distinguish between used and unused lancets creates significant health and safety problems in which contaminated lancets may be used and then replaced into the storage bag among unused lancets for re-use without the knowledge of the next user. Given the many prevalent blood-borne diseases such as hepatitis, a single such misidentification is potentially catastrophic. Additionally, federal regulations and requirements of the health care industry, such as Medicare, contain provisions that medical and heath providers cannot use or stock returned lancet inventory without definite certainty that the returned items have not been used or tampered with.

It can therefore be appreciated that a need exists for an improved lancet design that overcomes the foregoing problems associated with visual and tactile misidentification of used lancets. The present invention addresses such needs.

### SUMMARY OF THE INVENTION

An improved protective lancet, lancet casing and method for producing the same are disclosed herein. The lancet casing includes a lancet body that is integrally molded about an elongated shaft portion of a lancet needle. The lancet body is characterized in having an upper bearing surface from which a pointed end of the lancet needle extends. The lancet casing further includes a cap integrally molded about the pointed end of the lancet needle and joined to the lancet body by a narrow frangible junction on the upper bearing surface of the lancet body. In accordance with the present invention, one or more tab members are affixed between the cap and the body. Each of the one or more tab members includes a persistently affixed upper end integrally molded to the outer contour of the cap and a detachably affixed lower end integrally molded to or near a peripheral edge of the upper bearing surface of the lancet body to provide a readily perceptible user indication of the status of whether or not the cap member has been re-attached or tampered with.

The above as well as additional objects, features, and advantages of the present invention will become apparent in the following detailed written description.

### BRIEF DESCRIPTION OF THE DRAWINGS

The novel features believed characteristic of the invention are set forth in the appended claims. The invention itself however, as well as a preferred mode of use, further objects and advantages thereof, will best be understood by reference to the following detailed description of an illustrative embodiment when read in conjunction with the accompanying drawings, wherein:

FIG. 1 illustrates an improved lancet unit and lancet casing as manufactured prior to usage in accordance with one embodiment of the present invention;

FIG. 2 depicts the improved lancet unit shown in FIG. 1 with the lancet cap detached;

FIG. 3 illustrates the lancet unit depicted in FIG. 2 with the lancet cap re-attached;

FIG. 4 depicts an improved lancet unit and lancet casing as manufactured prior to usage in accordance with an alternate embodiment of the present invention;

FIG. 5 illustrates an improved lancet unit in accordance with an alternate embodiment of the present invention; and

FIGS. 6A-6B depicts alternate lancet tab dispositions for preventing tampering and reducing the required detachment force in accordance with alternate embodiments of the present invention.

### DETAILED DESCRIPTION OF A PREFERRED EMBODIMENT

This invention is described in a preferred embodiment in the following description with reference to the figures. While this invention is described in terms of the best mode for achieving this invention's objectives, it will be appreciated by those skilled in the art that variations may be accomplished in view of these teachings without deviating from the spirit or scope of the present invention.

The present invention is directed to a new type of protective lancet casing unit that provides readily perceptible evidence of prior lancet usage or tampering by providing an indication of whether or not the cap has been previously removed or partially detached. As explained in further detail with reference to the figures, an improved lancet and lancet casing are disclosed which provide such evidence of prior use or tampering by implementation of a boundary attachment, or tab member that attaches at one end to a lancet cap and at the other end to the lancet body. In accordance with the embodiments disclosed herein, the relative thickness of the molding used at the points of contact between the tab and the protective cap and the surface of the lancet is controlled such that by applying a thicker amount of molding at one of these points of contact and a thinner molding at the other, the attachment will consistently break-off from the thinner attachment. Thus, the user can routinely identify whether a detachment has occurred by visually inspecting the same location on each lancet.

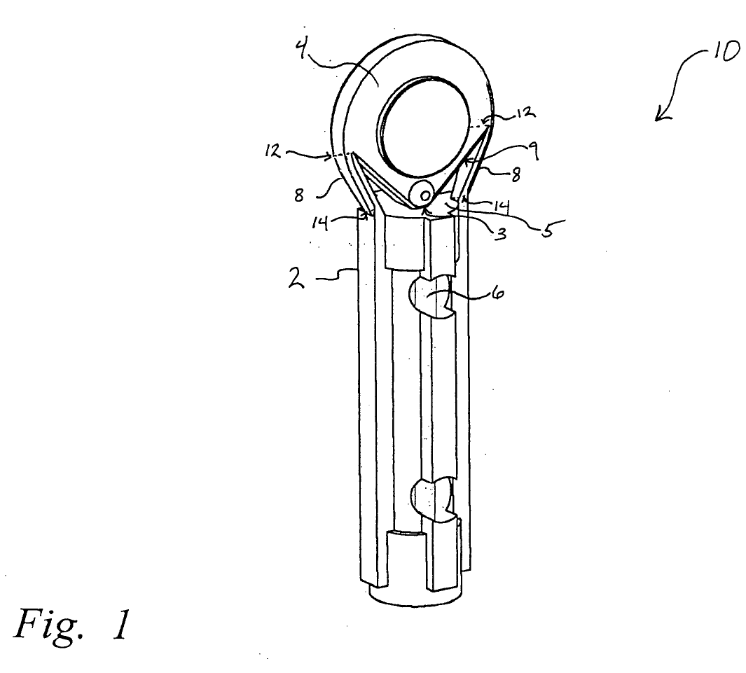

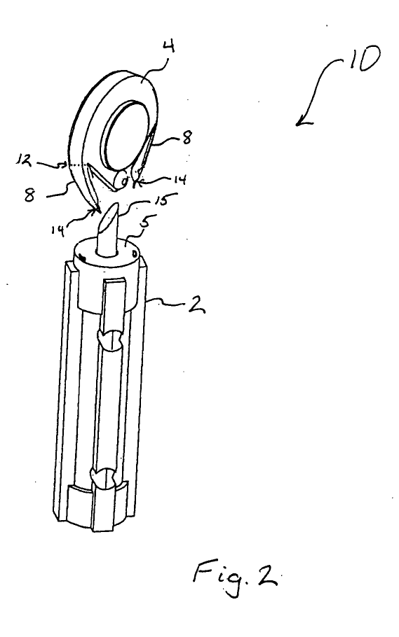

With reference to the figures, wherein like reference numerals refer to like and corresponding parts throughout, and in particular with reference to FIG. 1, there is illustrated an improved lancet unit having a lancet casing as manufactured prior to usage in accordance with one embodiment of the present invention. Specifically, a lancet unit 10 is depicted as generally including an outer encasement molded directly about a lancet needle which is comprised of a generally cylindrical elongated shaft portion 6 and having a pointed end section 15 depicted in FIG. 2. Consistent with conventional skin pricking applications for which it is designed, lancet unit 10 is a relatively small device having a length typically ranging from three to five centimeters.

Lancet unit 10 includes a lancet body 2 that is generally cylindrical in form with generally cylindrical end sections connected by a small diameter axial cylindrical section. The top portion of lancet unit 10 comprises a cap member 4 that is integrally and sealably joined to lancet body 2 by a frangible junction 3 that is preferably of a reduced diameter so that frangible junction 3 can be readily fractured to detach cap member 4 from an upper bearing surface 5 by exerting a twisting or pulling force, as by rotating cap member 4 relative to lancet body 2. In the depicted embodiment, cap member 4 has a rounded edge contour extending well beyond the outer diameter of lancet body 2. Furthermore, cap member 4 includes opposing face surfaces to provide a relatively flat or disc-like member that can be conveniently grasped between the ends of the thumb and forefinger when it is desired to rotatably detach cap member 4 relative to lancet body 2.

In a preferred embodiment, a plastic mold injection process is utilized to integrally join cap member 4 with lancet body 2 wherein cap member 4 is molded about the pointed end section 15 and lancet body 2 is molded about lancet needle shaft 6. In this manner, when cap member 4 is detached from lancet body 2, the pointed end section 15 is exposed, extending from the substantially flat upper bearing surface 5 of the lancet body. The pointed end section 15 may extend from the upper bearing surface 5 using any well-known lancet configurations, such as by having the needle protrude outward from the lancet body 2 or by otherwise affixing the needle to the upper bearing surface 5. It will be apparent from the foregoing description of the embodiment depicted in the figures that the lancet encasement comprised of lancet body 2 and cap member 4 serves both as an integral support to hold lancet unit 10 prior to and during use and as a container to protectively and sealably enclose pointed end section 15 until lancet unit 10 is ready for use. In addition to providing a grippable body by which lancet unit 10 can be maneuverably handled, the flat upper bearing surface 5 of lancet body 2 serves as a bearing for limiting the depth of penetration of the lancet needle to a few millimeters into human skin.

The pointed end section 15 of the lancet needle is completely enclosed and sealed by cap member 4, which is formed integrally with lancet body 2 and sealably joined therewith so that the pointed end is protected from external sources of contamination prior to use. During use, cap member 4 is detached from lancet body 2 to expose the pointed end section 15 of the lancet needle which is then used to pierce a patient's skin either manually by hand pressing the lancet or as deployed in a mechanically driven lancet device. The lancet and protective cap may be manufactured using one or more of a variety of suitable materials such as semi-rigid plastics, which are suitable for manufacturing using well-known injection molding manufacturing techniques. Likewise the needle may be of any type of material known in the art, such as stainless steel.

In accordance with the present invention, lancet unit 10 further includes an additional attachment means usefully deployed as an integrally adapted member of the outer encasement comprising cap member 4 and lancet body 2. Specifically, and as illustrated in FIG. 1, a pair of tab members 8 are disposed between cap member 4 and the upper/outer edge of lancet body 2. In the depicted embodiment, each of tab members 8 are plastic strips which taper inward from a wider tab base 12 integrally joined with the side edge of cap member 4 to a narrower tear-away end 14 affixed to upper bearing surface 5. The relative width of tab base 12 ensures that responsive to a twisting removal action, tab member 8 will remain attached at its upper end to cap member 4 while the lower tear-away joint 14 will disengage from upper bearing surface 5 of lancet body 2 substantially simultaneously with fracturing of frangible junction 3.

In an alternative embodiment, each of tab members 8 are plastic strips which taper outward from a narrower tear-away end affixed to the side edge of cap member 4 to a wider tab base integrally joined with the upper bearing surface 5. The relative width of the tab base ensures that responsive to a twisting removal action, tab member 8 will remain attached at its lower end to the upper bearing surface 5 of lancet body 2 while the upper tear-away joint will disengage from cap member 4 substantially simultaneously with fracturing of the frangible junction.

As further depicted in FIG. 1, each of tab members 8 is attachably disposed such that a substantial gap is formed between the edge of cap member 4 and the extended strip portion of each of tab members 8 between tab base 12 and tear-away end 14. Furthermore, tab members 8 are affixed in mutual diametric opposition with tab members 8 disposed on the outer contour edge of cap member 4 to facilitate ease of disengagement of cap member 4 by providing a balanced rotational lever effect. In the depicted embodiment, wherein cap member 4 has side edges extending beyond the axial diameter of lancet body 2 and tab members 8 attached thereto at tab bases 12 and extending inwardly toward the attachment points on upper bearing surface 5, a twisting force on cap member 4 is multiplied as applied on the narrower diameter of the points of attachment of tear-away ends 14 on upper bearing surface 5 thereby facilitating ease of disengagement of tab members 8.

FIG. 2 depicts the improved lancet unit 10 shown in FIG. 1 with the lancet cap member 4 disengaged from lancet body 2. As shown in FIG. 2, when cap member 4 is detached from lancet body 2, an exposed pointed end section 15 extends a short distance from upper face 5, which provides a depth control bearing surface. The pointed end section 15 of lancet unit 10 can be formed in any suitable manner but preferably is provides with one or more sharp cutting edges which will effectuate bleeding from the capillaries immediately below the surface of the skin for drawing a small drop of blood from a patient's finger or other suitable piercing site.

Detachment of cap member 4 from lancet body 2 is effectuated by fracturing its base contact at frangible junction 3 immediately surrounding the base perimeter of pointed end section 15 while substantially simultaneously breaking the molded joint between the tear-away end 14 and the upper bearing surface 5. Such detachment is typically effectuated by a hand twisting motion in which the suitably disk shaped cap member 4 is grasped between a user's thumb and index finger while the lancet body 2 is grasped by the fingers of the user's other hand. In the depicted embodiment, this detachment technique is effective for the flat-faced, disk like cap member 4 by providing a well-suited lever for applying sufficient force to simultaneously detach the pair of tab members 8 from upper bearing surface 5 of the lancet body. In a preferred embodiment, and as shown in FIG. 2, tab members 8 disengage from lancet body 2 at or near the lower tear-away ends 14 such that a minimum amount of plastic molding residue remains which will not appreciably affect the shape or contour of lancet body 2 thus preventing any potential interference by such residue with upper bearing surface 5 or with the fitting of the uncapped lancet into a lancing device. The minimal amount of plastic mold material remaining on upper lancet body face 5 after detachment of cap member 4 may usefully provide a post re-attachment visual indicator that cap member 4 has previously been detached from lancet body 2.

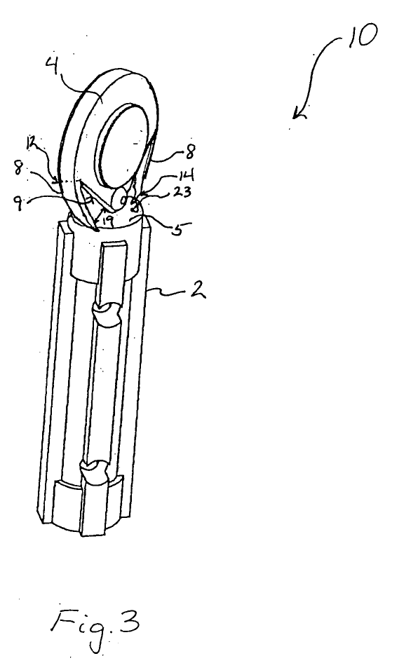

With reference to FIG. 3, there is illustrated the lancet unit depicted in FIG. 2 with the cap member 4 re-attached to lancet body 2. As shown in FIG. 3, the edge contour of cap member 4 includes tapered side edges 9 from which a substantial gap 19 extends therebetween to tab members 8. Gaps 19 facilitate unobstructed viewing of the upper bearing surface 5 both when cap member 4 is originally attached or re-attached to lancet unit 10. Specifically, the gaps 19 between each of tapered side edges 9 and the medial strip portions of tab members 8 affords an unobstructed and open view at virtually any perspective viewing angle of the points on upper bearing surface 5 at which tear-away ends 14 are or were moldedly affixed to reveal the present condition of the tear-away ends 14 as either attached to or disengaged from upper bearing surface 5. It should be noted that although tapered edge 9 is depicted in the disclosed embodiments as a straight line taper, alternative taper angles such as a concave taper wherein tapered edge 9 slopes at an inward curvature toward the central axis of cap member 4 may be employed consistent with the spirit and scope of the present invention.

As depicted in FIG. 3, visually perceptible evidence of the original disengagement of cap member 4 such as a stretched, jagged, and/or twisted appearance of tear-away ends 14, and the structural disengagement of tear-away ends 14 from their original attachment points 23 on upper bearing surface 5 are all readily perceptible with the open view forum provided by gaps 19. In this manner, the mutual orientation of tab members 8 with respect to cap member 4 and lancet body 2 provides a user with a greatly enhanced opportunity to visually inspect whether or not tab members 8 have been detached from the lancet body 2. In the alternative, the gaps 19 provide a user a tactile indicator wherein the user may push either of tab members 8 inward toward the tapered edge 9 of cap member 4 to ascertain whether or not tear-away end 14 remains fixed to lancet body 2.

The embodiment set forth FIGS. 1-3 further illustrates tab members 8 as having tab base 12 affixed at the edge of cap member 4 at the point where the tapered edge 9 of cap member 4 begins with tab members 8 extending tangentially downward from this point to the upper bearing surface 5 of lancet body 2. This arrangement increases the user's ability to inspect the tab both visually and by touch to determine whether it has been previously detached. In addition, tab members 8 may be formed of a color in contrast with the color of lancet body 2 to further aid in visually identification of the discontinuity between tab members 8 and lancet body 2. While the preferred embodiment includes the use of two diametrically opposed tab members, alternate implementations of the present invention may be implemented in which a single tab member or more than two are similarly deployed.

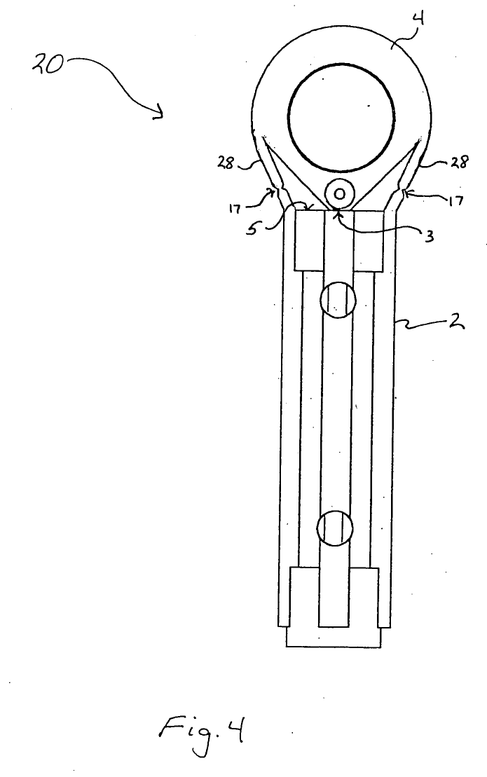

Referring to FIG. 4, there is depicted an improved lancet unit and lancet casing as manufactured prior to usage in accordance with an alternate embodiment of the present invention. Specifically, a lancet unit 20 is illustrated, which like the embodiment depicted in FIGS. 1-3 includes cap member 4 integrally molded to a lancet body 2. In contrast to the embodiment depicted in FIGS. 1-3, lancet unit 20 includes a pair of integrally molded tab members 28 each having a medial tear-away joint 17 disposed between the points at which each of tab members 28 attaches to cap member 4 and lancet body 2. As shown in FIG. 4, medial tear-away joint 17 comprises a substantially narrowed neck junction that will more readily fracture upon application of a twisting or pulling force applied between cap member 4 and lancet body 2. In this manner, when cap member 4 is detached from lancet body 2, a substantial appendage portion of the original tab member 8 remains attached to cap member 4 while a visually perceptible stub remains the upper bearing surface 5 of lancet body 2. Medial tear-away joint 17 is preferably disposed within one to three millimeters from the attachment to upper bearing surface 5 such that the remaining stub provides a readily perceptible indicator of de-attachment while not unduly interfering with the surface contour of upper bearing surface 5.

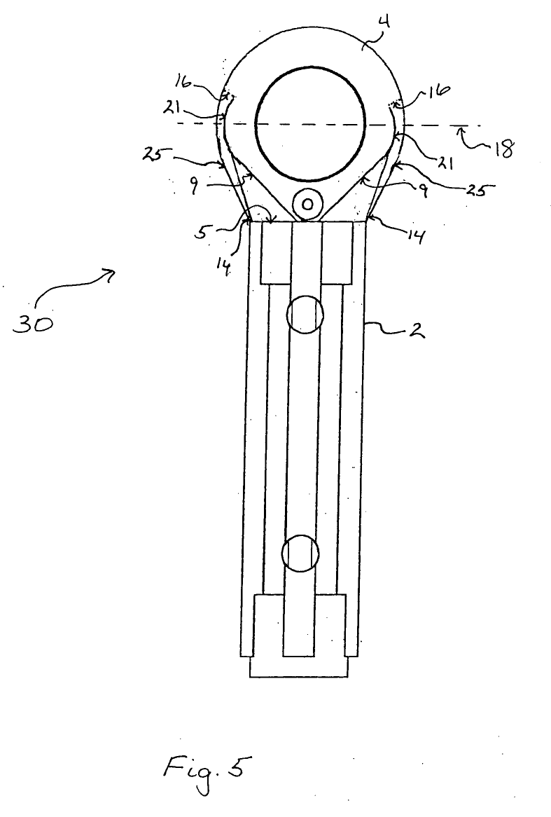

With reference to FIG. 5, there is illustrated an improved lancet unit 30 in accordance with an alternate embodiment of the present invention. Specifically, lancet unit 30 includes a pair of tab members 25 that are persistently affixed at respective tab bases 16 above a fulcrum boundary 18 on the curved edge contour of cap member 4. As utilized herein, “fulcrum boundary” refers to the outermost point at or near the diametric center axis of a disc-shaped or partially disc-shaped cap member 4 such as the partially disc-shaped cap members depicted in FIGS. 1-5. As shown in the depicted embodiment, each of tab members 25 is integrally attached to cap member 4 only at the molded junctions comprising tab bases 16 from which the tab members extend downward and inward toward their respective attachment point on upper bearing surface 5. The downward and inward extension of tab members 25 from tab bases 16 results in an upper portion of each of tab members 25 resting or pressing in non-adhered surface contact against the curved outer edge contour of cap member 4 along a pair of diametrically opposed non-adhered boundaries 21. With tab members 25 moldedly attached at its ends between cap member 4 and lancet body 2, the non-adhered boundaries 21 formed between tab members 25 and the convex curved outer contour of cap member 4 extends from tab base 16 to the point below fulcrum boundary 18 at which each of tab members 25 departs tangentially from the edge surface of cap member 4 wherein tab members 25 extend downwardly and inwardly to their respective attachment points on upper bearing surface 5 of lancet unit 30.

In accordance with the depicted embodiment, tear-away ends 14 are detachably affixed by plastic molding or another suitable attachment technique such that tab members 25 are pressed against the surface of cap member 4 along non-adhered boundary 21. Upon detachment of cap member 4 from lancet body 2 wherein tear-away ends 14 are disengaged from upper bearing surface 5, the released tear-away ends 14 of tab members 25 are urged outward as and upward by the tension maintained at tab bases 16 as applied at the fulcrum boundary 18. In this manner, upon re-attachment of cap member 4 to lancet body 2, tab members 25 are slightly splayed further enabling a user to readily make a reliable visual determination that cap member 4 has been previously detached.

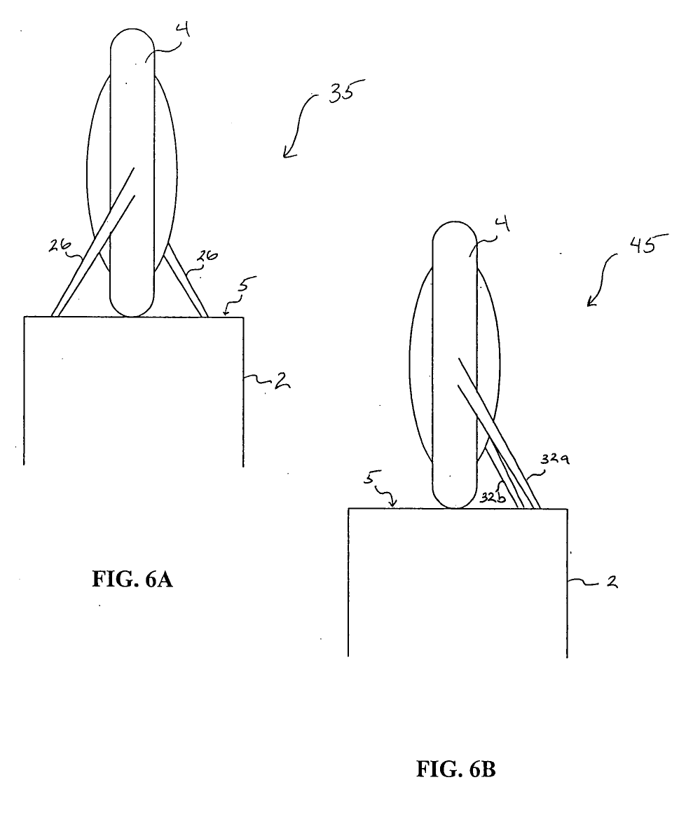

FIGS. 6A and 6B depict alternate lancet tab dispositions for preventing tampering and reducing the required detachment force in accordance with alternate embodiments of the present invention. Specifically, FIG. 6A illustrates an improved lancet unit 35 having a pair of tab members 26, which similar to tab members 8 of lancet unit 10, are persistently affixed at an upper end to the side edge of disc-like cap member 4 and detachably affixed a lower end to upper bearing surface 5. As shown in FIG. 6A, tab members 26 are pre-set in a “pre-twisted” disposition in which each of tab members 26 extends angularly from its attachment point on cap member 4 to the corresponding attachment points on upper bearing surface 5. In the depicted embodiment, the angled extension of tab members 26 reduces the required rotation distance required to impart a tab fracturing force thus facilitating ease of disengagement of the tab members 26 from upper bearing surface upon twisting cap member 4 in a counterclockwise direction with respect to upper bearing surface 5.

Referring to FIG. 6B, a lancet unit 45 is illustrated in which each of a pair of tab members 32 are pre-set in mutually opposing “pre-twisted” dispositions with tab member 32a angled to facilitate ease of detachment in the clockwise direction and with tab member 32b angled to facilitate ease of detachment in the counterclockwise direction. In this manner, one or the other of tab members will be disengaged from upper bearing surface 5 upon even the slightest twist of cap member 4 in either direction thus providing enhanced tamper resistance for lancet unit 45.

While this invention has been described in terms of several embodiments, it is contemplated that alterations, permutations, and equivalents thereof will become apparent to one of ordinary skill in the art upon reading this specification in view of the drawings supplied herewith. It is therefore intended that the invention and any claims related thereto include all such alterations, permutations, and equivalents that are encompassed by the spirit and scope of this invention.

What is claimed is:

1. A protective lancet casing comprising:
a lancet body having an upper surface from which a pointed end of a lancet needle extends;
a cap member abutting the upper surface of said lancet body for encasing the pointed end of the lancet needle;
at least one tab member affixed at an upper end to said cap member and affixed at a lower end to said lancet body;
wherein said at least one tab member is permanently affixed at the upper end to said cap member and permanently affixed at the lower end to said lancet body, and wherein said at least one tab member includes a detachable junction between the upper and lower ends.

2. The protective lancet casing of claim 1, wherein said at least one tab member is tapered such that the upper end forms a wider molded junction with said cap member and the lower end forms a wider molded junction with said lancet body and wherein said at least one tab member includes a narrower molded detachable junction between the upper and lower ends.

3. The protective lancet casing of claim 1, wherein the upper surface of the lancet body is substantially flat for providing a bearing surface with respect to the pointed end of the lancet needle.

4. The protective lancet casing of claim 1, wherein said lancet body, said cap member, and said at least one tab member are integrally molded as a single unit by a mold injection process.

5. The protective lancet casing of claim 1, wherein said cap member has an outer contour that tapers inward toward the upper surface of said lancet body such that a substantial gap is formed between the tapered contour and the lower end of the at least one tab member.

6. A protective lancet casing comprising:
a lancet body having an upper surface from which a pointed end of a lancet needle extends;
a cap member abutting the upper surface of said lancet body for encasing the pointed end of the lancet needle;
at least one tab member affixed at an upper end to said cap member and affixed at a lower end to said lancet body;
wherein said at least one tab member includes two tab members attached in diametric opposition on said lancet body.

7. The protective lancet casing of claim 6, wherein each of the two diametrically opposing tab members are pre-disposed at opposite twist angles to facilitate detachment of the lower end of one of the two tab members in a clockwise twist direction and detachment of the lower end of the other of the two tab members in a counterclockwise twist direction.

8. The protective lancet casing of claim 6, wherein each of the two diametrically opposing tab members are pre-disposed at a twist angle to facilitate ease of detachment of the respective lower ends of both of the two tab members upon twisting in one twist direction.

9. The protective lancet of claim 8, wherein the twist angle of both of the two diametrically opposed tab members facilitates twisting detachment in either the clockwise or counterclockwise direction with respect to the upper surface of said lancet body.

10. A lancet unit comprising:
an integrally molded plastic encasement housing a lancet needle, wherein said encasement includes:
a body member molded about a shaft portion of the lancet needle, said body member including an upper bearing surface from which a pointed end section of the lancet needle protrudes;
a cap member molded about the pointed end section of the lancet needle; and
at least one plastic strip member extending between said cap member and said body member, said at least one plastic strip member affixed at an upper end to said cap member and affixed at a lower end at or near the periphery of said upper bearing surface such that a visually perceptible gap is formed between said lower end of said at least one plastic strip member and said cap member;
wherein the upper end of the at least one plastic strip member is integrally affixed above a fulcrum boundary point on a curved outer contour of said cap member such that an upper portion of the at least one plastic strip member is in non-adhered surface contact with a portion of the curved outer contour of said cap member.

11. The lancet unit of claim 10, wherein the at least one plastic strip member departs tangentially from the non-adhered surface contact with the curved outer contour of said cap member.

12. The lancet unit of claim 10, wherein said at least one plastic strip member comprises two plastic strip members.

13. The lancet unit of claim 12, wherein each of the two plastic strip members are pre-disposed at opposite twist angles to facilitate detachment of the lower end of one of the two plastic strip members in a clockwise twist direction and detachment of the lower end of the other of the two plastic strip members in a counterclockwise twist direction.

14. The lancet unit of claim 12, wherein each of the two plastic strip members are pre-disposed at a twist angle to facilitate ease of detachment of the respective lower ends of both of the two plastic strip members upon twisting in one twist direction.

15. The lancet unit of claim 14, wherein the twist angle of both of the two plastic strip members facilitates twisting detachment in either the clockwise or counterclockwise direction with respect to the upper surface of said body member.
